(19) United States
(12) Patent Application Publication
(10) Pub. No.: US 2022/0011309 A1
(43) Pub. Date: Jan. 13, 2022

(54) METHOD FOR THE RAPID IDENTIFICATION OF COVID-19 INFECTION

(71) Applicants: ARIZONA BOARD OF REGENTS ON BEHALF OF ARIZONA STATE UNIVERSITY, Scottsdale, AZ (US); MARV ENTERPRISES LLC, Hermitage, PA (US)

(72) Inventors: Qiang Chen, Chandler, AZ (US); Mitchell S. Felder, Hermitage, PA (US)

(21) Appl. No.: 17/370,514

(22) Filed: Jul. 8, 2021

Related U.S. Application Data
(60) Provisional application No. 63/049,441, filed on Jul. 8, 2020.

(51) Int. Cl. G01N 33/569; G01N 33/68

(52) U.S. Cl. CPC G01N 33/56983; G01N 33/6848; G01N 2469/10; G01N 2333/165; G01N 33/6854

(57) ABSTRACT

An embodiment provides a method for identifying Covid-19 in a body fluid of a patient, including: removing the body fluid from a patient; applying a treatment to the body fluid, wherein the treatment comprises an antibody that joins with a Covid-19 targeted antigen (TA) in the body fluid to form an antibody-TA complex, wherein the antibody comprises a light functional antibody; identifying the antibody-TA complex, using a spectroscopic technique, from the body fluid using a light source; and returning the body fluid to the patient. Other aspects are described and claimed.

### CROSS REFERENCE TO RELATED APPLICATION

This application claims priority to U.S. Provisional Patent Application Ser. No. 63/049,441, filed on Jul. 8, 2020, and entitled “METHOD FOR THE RAPID IDENTIFICATION OF COVID-19 INFECTION,” the contents of which are incorporated by reference herein.

### FIELD

This application relates generally to a method for the rapid identification of COVID-19, and, more particularly, to a fluorescent or luminous virion-antibody complex for the rapid identification of COVID-19.

### BACKGROUND

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is presently known as the severe acute respiratory syndrome coronavirus 2 (SARS-CoV-2). The disease it causes is called coronavirus disease 2019 (COVID-19).

Cases of COVID-19 have been reported in multiple countries, where it has caused a great deal of morbidity and mortality, in a worldwide pandemic. The disorder is characterized by shortness of breath, increased mucus production, sore throat, cough, and fever. This may necessitate admission to a hospital, with subsequent admission to an intensive care unit for the respiratory support of the infected patient.

There is a need for the rapid identification of Covid-19 from a patient's body fluid or sample (mucus, saliva, blood, cerebrospinal fluid), due to the worldwide pandemic of this infection.

### BRIEF SUMMARY

In summary, one embodiment provides a method for identifying Covid-19 in a body fluid of a patient, comprising: removing the body fluid from a patient; applying a treatment to the body fluid, wherein the treatment comprises an antibody that joins with a Covid-19 targeted antigen (TA) in the body fluid to form an antibody-TA complex, wherein the antibody comprises a light functional antibody; identifying the antibody-TA complex, using a spectroscopic technique, from the body fluid using a light source; and returning the body fluid to the patient.

Another embodiment provides a device for identifying Covid-19 in a body fluid of a patient, comprising: a transparent first stage including an inlet for the body fluid and at least one exterior wall defining a treatment chamber; a transparent second stage, fluidly connected to the first stage, comprising a removal module and an outlet for the body fluid, wherein the treatment chamber comprises a delivery tube for introducing an antibody into the treatment chamber, wherein the delivery tube comprises a hollow tube including at least one interior wall defining a plurality of holes through which the antibody can be added to the treatment chamber, wherein the treatment is delivered through the hollow tube in counter-current mode with reference to the body fluid; and at least one sensor to identify the light functional antibody; the device being configured to: remove the body fluid from a patient; apply a treatment to the body fluid, wherein the treatment comprises an antibody that joins with a Covid-19 targeted antigen (TA) in the body fluid to form an antibody-TA complex, wherein the antibody comprises a light functional antibody; identify the antibody-TA complex, using a spectroscopic technique, from the body fluid using a light source; and return the body fluid to the patient.

A further embodiment provides a method for identifying Covid-19 in a body fluid of a patient, comprising: removing the body fluid from a patient; applying a treatment to the body fluid, wherein the treatment comprises an antibody that joins with a Covid-19 targeted antigen (TA) in the body fluid to form an antibody-TA complex, wherein the antibody comprises a light functional antibody; identifying the antibody-TA complex, using a spectroscopic technique, from the body fluid using a light source; capturing, using an antibody microarray, the antibody-TA complex, wherein the microarray comprises a plurality of the antibody on a transparent solid surface; and returning the body fluid to the patient.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example partial cross-sectional view of a cylinder and tubing used to deliver a treatment to a bodily fluid.

FIG. 2 illustrates an example a partial cross-sectional view showing additional detail of the cylinder and tubing of FIG. 1.

FIG. 3 illustrates an example flow diagram of a method for identification of Covid-19.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

COVID-19 has spread worldwide and become a global pandemic. The loss of life, suffering, and economic struggles have reached all corners of the globe. Symptoms may manifest about 2-14 days after exposure. The symptoms may include fever, chills, cough, shortness of breath, difficulty breathing, fatigue, muscle/body aches, new loss of taste/smell, sore throat, congestion, runny nose, nausea, vomiting, or diarrhea. More severe symptoms may include trouble breathing, persistent pain/pressure in the chest, confusion, inability to wake or stay awake, or bluish lips/face. Some cases may require hospitalization and even intensive care unit healthcare. Because of the novelty of the virus, very few tests exist that are specific for COVID-19. What is needed is a method for rapid identification of COVID-19 in a patient.

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is known as the severe acute respiratory syndrome coronavirus 2 (SARS-CoV-2). The disease it causes is called coronavirus disease 2019 (COVID-19).

In a phylogenetic analysis of 103 strains of SARS-CoV-2 from China, two different types of SARS-CoV-2 were identified, designated type L (accounting for 70 percent of the strains) and type S (accounting for 30 percent). The strains in L type, derived from S type, are evolutionarily more aggressive and contagious.

Cases of COVID-19 have been reported in multiple countries, where it has caused a great deal of morbidity and mortality, in a worldwide pandemic. The disorder is characterized by shortness of breath, increased mucus production, sore throat, cough, and fever. This may necessitate admission to a hospital, with subsequent admission to an intensive care unit for the respiratory support of the infected patient. There is therefore a need for rapid identification of Covid-19.

Accordingly, described herein is a method for the rapid identification of Covid-19 from a patient's body fluid or sample, for example, blood, CSF (cerebrospinal fluid), mucus, or saliva. The method includes removing the body fluid from a patient which may contain Covid-19 virions, exposing the body fluid to at least one binding antibody so that any Covid-19 virions present in the body fluid form a virion antibody complex with the binding antibody, and then determining the presence or absence of the virion binding antibody complex. The binding antibody can include, for example, a fluorescent antibody, a luminous antibody, or combinations thereof. The binding antibody may be referred to as a light functional antibody. The concentration of binding antibody may be made as high as necessary for the identification of extremely small, e.g., picogram/microliter, concentrations of the final virion binding antibody complex. The signal from the virion binding antibody complex can be amplified as needed to identify a signal

The illustrated example embodiments will be best understood by reference to the figures. The following description is intended only by way of example, and simply illustrates certain example embodiments.

A method comprises treating a patient's body fluid with at least one binding antibody that reacts with and binds at least one antigen/target antigen (TA) of Covid-19. The body fluid can include any body tissue capable of containing the Covid-19 virus but is preferably selected from the group consisting of saliva, mucus, blood, and CSF. The binding antibody can include an antibody capable of detection using a spectroscopic technique, such as but not limited to, a fluorescent antibody, a luminous antibody, or combinations thereof. The Covid-19 antigen/TA can include, but is not limited to: Covid-19 spike glycoprotein, Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex forming virion antibody complexes, and combinations thereof. Identification of Covid-19 positivity is then made by a standard technique well known by laboratory personnel.

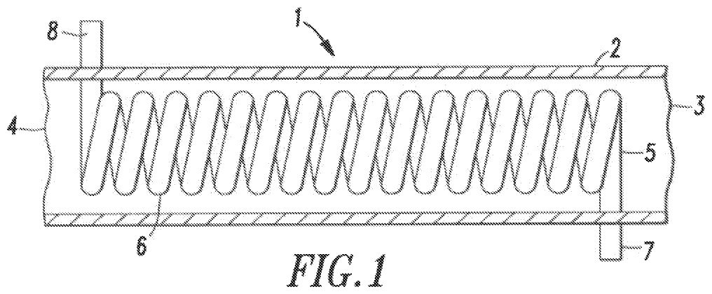

Referring to FIG. 1, the first stage can include an exterior wall 2 defining a treatment chamber 5. The treatment can be applied in the treatment chamber 5. Residence times of the blood to be treated can be altered by changing the dimensions of the treatment chamber or the flow rate of the body fluid through the treatment chamber 5. body fluid to be treated enters the inlet 3, passes through the treatment chamber 5, and exits the outlet 4. In embodiments, the treatment or antibodies can be applied from a delivery tube 6 located within the treatment chamber 5. An interior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the body fluid. As shown, the delivery tube 6 comprises a helical coil.

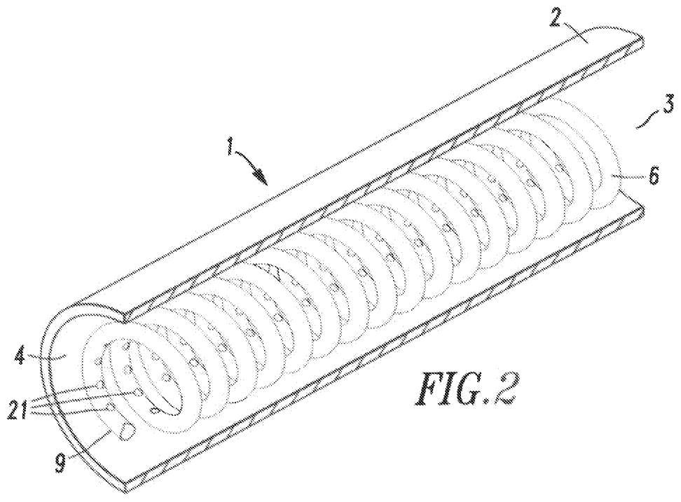

Referring to FIG. 2, the delivery tube 6 can be hollow and the interior wall 9 can define a plurality of holes 21. In this design for example, designer antibodies can be pumped through the delivery tube 6 to affect a desired concentration of antibodies in the body fluid (blood and/or CSF). The antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic and/or transparent.

The antibody, targeting the antigen: can be delivered in a concurrent or counter-current mode with reference to the blood and/or CSF. In counter-current mode, the body fluid enters the treatment chamber 5 at the inlet 3. The designer antibody can enter through a first lead 8 near the outlet 4 of the treatment chamber 5. The body fluid then passes to the outlet 4 and the designer antibodies pass to the second lead 7 near the inlet 3. The removal module of the second stage substantially removes the designer antibodies-antigen molecular compound from the blood and/or CSF.

In the first stage, a body fluid may be withdrawn from a patient using standard medical techniques, such as, but not limited to a sterile cotton swab, blood draw, or lumbar puncture. Other techniques known to those skilled in the art are contemplated by this disclosure.

In the second stage a treatment may be applied to the body fluid. The treatment comprises exposing the body fluid to a binding antibody, such as F/LT Ab, that binds to an antigen specific to Covid-19. The Covid-19 specific antigen can include at least one antigen selected from the group consisting of Covid-19 spike glycoprotein, Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp?-nsp8 complex, nsp10-nsp14 complex, nsp10-nsp16 complex, and combinations thereof. The binding antibody and Covid-19 specific antigen form a virion antibody complex (F/LT Ab-TPA complex).

The virion antibody complex can be detected by standard laboratory techniques, such as spectroscopy. The amplified signal from the laboratory technique created by the virion antibody complex can be transmitted to a control unit which identifies the Covid-19 positivity or negativity. The entire system may be monitored and controlled utilizing a computer. Persons having ordinary skill in art will recognize that the steps described above can be performed on various devices/machines.

As an alternative to using a type of device as described above, the Covid-19 virions may be captured using antibody microarrays containing a binding antibody in a microarray. In one embodiment, the binding antibody comprises a fluorescent antibody (Fl). In a second embodiment, the binding antibody comprise a luminescent (Lu) antibody. The binding antibody can be organized in a microarray. The microarray is a plurality of antibodies fixed on a solid surface. The solid surface can be any suitable material but is conveniently transparent and selected from a group consisting of glass, plastic, silicon, and combinations thereof. The microarray allows detection of the virion antibody complex.

The microarray may comprise a plurality of monoclonal antibodies attached at high density on the solid surface. Typically, the microarray will contain millions of antibodies, and the solid surface is transparent to facilitate detection. Any microarrays known by those skilled in the art sufficient to perform the described technique and are anticipated by this disclosure.

Exposure of the virion to the binding antibodies on the microarray may create the virion antibody complex. The complex can be tracked using an appropriate sensor. All steps in the process can be monitored and controlled by a computer in real time. Persons having ordinary skill in art will recognize that the steps described above can be performed on various devices/machines. This disclosure contemplates all known devices/machine that can perform the steps described in the above illustrative example.

To identify the virion antibody complex after exposure in the microarrays, the body fluid may then be forced through a container preferably constructed from a transparent material, which exposes the virion antibody complex to a light-sensing device. The sensing device also creates an enlarged, magnified visual image of virion antibody complex.

A concentrated and focused intense energy beam, such as light, may then be used to properly illuminate the virion antibody complex within the body fluid. Each virion antibody complex may be rapidly identified. The virion antibody complex may also be identified and tracked using optical or digital enhancement or magnification. Location and tracking of the virion antibody complex may also be achieved using computer graphics and computer programs well known in the art. Latency times can be less than one microsecond. An alternative methodology would use optical pattern recognition of the virion antibody complex. Persons having ordinary skill in art will recognize that the steps described above can be performed on various devices/machines. This disclosure recognizes all known devices/machine that can perform the steps of the method.

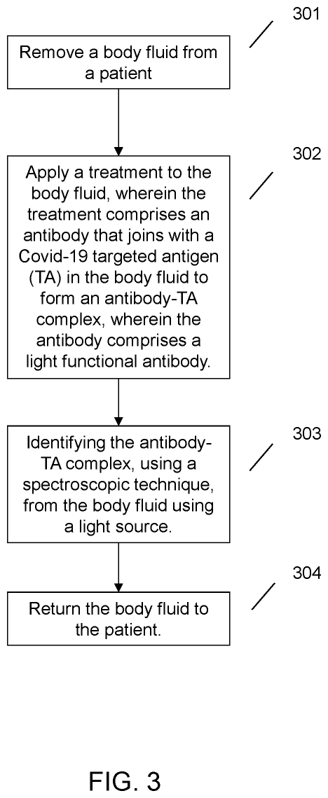

Referring to FIG. 3, an example method is illustrated. A method for treating a body fluid comprising: a first stage including removing the body fluid from a patient at 301; a second stage including applying a treatment to the body fluid wherein the treatment comprises an antibody that joins with an Covid-19 targeted antigen (TA) in the body fluid to form an antibody-TA complex, wherein the treatment comprises an antibody that joins with a Covid-19 targeted antigen (TA) in the body fluid to form an antibody-TA complex, wherein the antibody comprises a light functional antibody at 302, and identifying the antibody-TA complex, using a spectroscopic technique, from the body fluid using a light source at 303; and, as an optional step a third stage including returning the body fluid to the patient at 304.

An exemplar embodiment has been described above. However, it will be apparent to those skilled in the art that numerous variations of the type described could be made to the present invention without departing from the spirit of the invention. The scope of the present invention is defined by the broad general meaning of the terms in which the claims are expressed.

Unless otherwise indicated, all numbers expressing quantities of ingredients, properties such as molecular weight, reaction conditions, and so forth used in the specification and claims are to be understood as being modified in all instances by the term “about.”

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

While the invention has been particularly shown and described with reference to a preferred embodiment, it will be understood by those skilled in the art that various changes in form and detail may be made therein without departing from the spirit and scope of the invention. All documents, books, manuals, papers, patents, published patent applications, guides, abstracts, and other references cited herein are incorporated by reference in their entirety.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data.

Embodiments may be implemented as an instrument, system, method or program product. Accordingly, an embodiment may take the form of an entirely hardware embodiment, or an embodiment including software (including firmware, resident software, micro-code, etc.) that may all generally be referred to herein as a “circuit,” “module” or “system.” Furthermore, embodiments may take the form of a program product embodied in at least one device readable medium having device readable program code embodied thereon.

A combination of device readable storage medium(s) may be utilized. In the context of this document, a device readable storage medium (“storage medium”) may be any tangible, non-signal medium that can contain or store a program comprised of program code configured for use by or in connection with an instruction execution system, apparatus, or device. For the purpose of this disclosure, a storage medium or device is to be construed as non-transitory, i.e., not inclusive of signals or propagating media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand-held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for identifying Covid-19 in a body fluid of a patient, comprising:
removing the body fluid from a patient;
applying a treatment to the body fluid, wherein the treatment comprises an antibody that joins with a Covid-19 targeted antigen (TA) in the body fluid to form an antibody-TA complex, wherein the antibody comprises a light functional antibody; and
identifying the antibody-TA complex, using a spectroscopic technique, from the body fluid using a light source.

2. The method of claim 1, wherein the Covid-19 targeted antigen is selected from the group consisting of: Covid-19 spike glycoprotein, Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp?-nsp8 complex, nsp10-nsp14 complex, and nsp10-nsp16 complex.

3. The method of claim 1, wherein the light functional antibody comprises a luminescent antibody.

4. The method of claim 1, wherein the light functional antibody comprises a fluorescent antibody.

5. The method of claim 1, wherein the identifying the antibody-TA complex comprises an intense energy beam to properly illuminate the antibody-TA complex.

6. The method of claim 1, wherein the identifying the antibody-TA complex comprises a control unit to determined Covid-19 positivity in the body fluid.

7. The method of claim 1, further comprising capturing the antibody-TA complex with an antibody microarray.

8. The method of claim 7, wherein the microarray comprises a plurality of the antibody on a solid surface.

9. The method of claim 8, wherein the solid surface comprises a transparent material.

10. The method of claim 1, wherein the identifying comprises at least one sensor to identify the light functional antibody.

11. A device for identifying Covid-19 in a body fluid of a patient, comprising:
a transparent first stage including an inlet for the body fluid and at least one exterior wall defining a treatment chamber;
a transparent second stage, fluidly connected to the first stage, comprising a removal module and an outlet for the body fluid, wherein the treatment chamber comprises a delivery tube for introducing an antibody into the treatment chamber, wherein the delivery tube comprises a hollow tube including at least one interior wall defining a plurality of holes through which the antibody can be added to the treatment chamber, wherein the treatment is delivered through the hollow tube in counter-current mode with reference to the body fluid; and
at least one sensor to identify the light functional antibody;
the device being configured to:
remove the body fluid from a patient;
apply a treatment to the body fluid, wherein the treatment comprises an antibody that joins with a Covid-19 targeted antigen (TA) in the body fluid to form an antibody-TA complex, wherein the antibody comprises a light functional antibody; and
identify the antibody-TA complex, using a spectroscopic technique, from the body fluid using a light source.

12. The device of claim 11, wherein the Covid-19 targeted antigen is selected from the group consisting of: Covid-19 spike glycoprotein, Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

13. The device of claim 11, wherein the light functional antibody comprises a luminescent antibody.

14. The device of claim 11, wherein the light functional antibody comprises a fluorescent antibody.

15. The device of claim 11, wherein the identifying the antibody-TA complex comprises an intense energy beam to properly illuminate the antibody-TA complex.

16. The device of claim 11, wherein the identifying the antibody-TA complex comprises a control unit to determined Covid-19 positivity in the body fluid.

17. The device of claim 11, further comprising capturing the antibody-TA complex with an antibody microarray.

18. The device of claim 17, wherein the microarray comprises a plurality of the antibody on a solid surface.

19. The device of claim 18, wherein the solid surface comprises a transparent material.

20. A method for identifying Covid-19 in a body fluid of a patient, comprising:
removing the body fluid from a patient;
applying a treatment to the body fluid, wherein the treatment comprises an antibody that joins with a Covid-19 targeted antigen (TA) in the body fluid to form an antibody-TA complex, wherein the antibody comprises a light functional antibody;
identifying the antibody-TA complex, using a spectroscopic technique, from the body fluid using a light source;
capturing, using an antibody microarray, the antibody-TA complex, wherein the microarray comprises a plurality of the antibody on a transparent solid surface; and
returning the body fluid to the patient.
